(19) United States
(12) Patent Application Publication
(10) Pub. No.: US 2002/0018512 A1
(43) Pub. Date: Feb. 14, 2002

(54) THIN BARRIER FILM FOR CONTAINMENT OF HALOGENATED AROMATIC COMPOUNDS IN A CHEMICAL THERMOMETER

(75) Inventors: RODNEY K. HEHENBERGER, APPLE VALLEY, MN (US); SARA L. MEGCHELSEN, ST. PAUL, MN (US); RICHARD L. JACOBSON, ST. PAUL, MN (US)

Correspondence Address: JOHN H HORNICKEL, 3M OFFICE OF INTL PROP COUNSEL, P O BOX 33427, ST PAUL, MN, 551333427

(73) Assignee: 3M Innovative Properties Company

(21) Appl. No.: 09/108,594

(22) Filed: Jul. 1, 1998

(51) Int. Cl. G01K 11/12; A61B 5/00; G01K 11/06; C08L 63/00

(52) U.S. Cl. 374/162; 523/440; 374/160; 600/549; 252/408.1; 252/962

(57) ABSTRACT

A barrier for halogenated aromatic compounds is provided, especially fluorinated hydrocarbon films useful for containing precise quantities of halogenated aromatic compounds used as thermally sensitive materials in reversible chemical thermometers. A new shape of chemical thermometer is also disclosed.

### FIELD OF THE INVENTION

This invention relates to barrier films for use to contain halogenated aromatic compounds, particularly such compounds as are used in chemical thermometers. This invention also relates to chemical thermometers generally and to the shape of such chemical thermometers.

### BACKGROUND OF INVENTION

Over the last 20 years, clinical thermometry has developed single-use, handheld thermometers that use precise quantities of different compositions of solid solutions of two thermally sensitive materials in a plurality of cavities on a single strip of laminated material, each composition in each cavity alterable at a different temperature. The precision of the different compositions responsive to the different temperatures has permitted these chemical thermometers to be truly qualified as clinical devices.

A number of patents describe the advances made to this field of clinical thermometry. Among these patents are those issued to Hof et al. such as U.S. Pat. Nos. 4,232,552; 4,299,727; 4,345,470; and 4,397,570, which describe improvements to the temperature indicating compositions of matter, the all-plastic construction of the thermometer, and the like.

Automated assembly of the clinical thermometer reduces costs, improves productivity, improves quality assurance, and provides other benefits. In the assembly of a clinical thermometer where as many as 45-50 different compositions are being deposited in a like number of separate cavities on the thermometer surface, precise metering and delivery of the compositions to the cavities, in registration, is essential for productive assembly of the clinical thermometers. U.S. Pat. No. 3,810,779 (Pickett et al.) discloses a method and apparatus for depositing precisely metered quantities of liquid on a surface, particularly temperature indicating compositions of matter on to the surface of a clinical thermometer. The method and apparatus disclosed in Pickett et al. has formed a basis for the construction of automated machinery to measure and deliver 45-50 different compositions to the like number of cavities for detecting temperatures ranging from 96.0° F. to 104.8° F. for the American market (35.5° C. to 40.4° C. for international markets).

Clinical thermometry has enjoyed the advances disclosed by the Hof et al. patents and the Pickett et al. patent because the thermometers can be precisely manufactured inexpensively and because the inexpensive clinical thermometers can be disposed of after a single use. The clinical thermometer can be used, then the instrument disposed to avoid the transmission of viruses, bacteria, and other germs that unfortunately plague reusable thermometer devices.

Clinical thermometry also is benefitting from advances in the nature of the temperature indicating compositions of matter whereby the amount of energy and time needed to reverse the “firing” of one of the compositions in one of the cavities from freezing temperatures overnight to cooling temperatures for a few hours. Some of these new reversible thermometer compositions employ an emulsion of a thermally sensitive material, means for observing a change in state of the material such as a dye, and a matrix forming material in which the thermally sensitive material is dispersed. Reversible thermometer compositions of this type are disclosed in European Patent Publication 0 684 463 (Al) (Hof) and related copending, coassigned, U.S. Pat. Appln. Ser. No. 08/425,162 (Hof) now allowed.

The chemical composition of the thermometer compositions, particularly the reversible thermometer compositions, are halogenated aromatic compounds that need to be contained within the plastic thermometer cavities to minimize any disruption to the fragile chemical balance and fragile physical balance of the thermally sensitive material in each cavity. Accuracy and precision require maintenance of such balances.

### SUMMARY OF INVENTION

What is needed in the art is a new type of barrier film to be used in the construction of the chemical thermometer or any other device containing a halogenated aromatic compound.

One aspect of the present invention is a barrier film for aromatic halogenated compounds, comprising a fluorinated hydrocarbon composition selected from the group consisting of fluorinated ethylene-propylene, perfluoroalkyl-tetrafluoroethylene, polyvinylidene fluoride, ethylene-chlorotrifluoroethylene, polychlorotrifluoroethylene, ethylene-tetrafluoroethylene, and combinations thereof.

Another aspect of the present invention is a chemical thermometer comprising (a) a base layer having a multiplicity of cavities defined therein to determine a like number of predetermined temperatures in a predetermined temperature range, the cavities containing a like number of different compositions of matter therein, (b) a transparent cover sheet in sealing engagement therewith, (c) a different thermometer composition in each cavity, and (d) a barrier film of a fluorinated hydrocarbon composition residing between the base layer and the thermometer composition in each cavity.

Another aspect of the present invention is a chemical thermometer, comprising a base layer having a multiplicity of cavities defined therein to determine a like number of predetermined temperatures in a predetermined temperature range, the cavities containing a like number of different compositions of matter therein, a transparent cover sheet in sealing engagement therewith, and a different thermometer composition in each cavity, wherein the thermometer has a handling portion and a sensing portion, and wherein the handling portion has substantially parallel sides.

A feature of the present invention is that the barrier film of the present invention provides a better (or more resistant chemical) barrier in a thinner film than previously used with commercial chemical thermometers. The barrier properties of the film as used in the present invention provides a tightly controlled environment for an aromatic chemical.

An advantage of the present invention is that the barrier film provides better containment of the aromatic compounds, particularly better containment of halogenated nitrobenzenes than previously found in typical barrier materials.

Another advantage of the present invention is that increased barrier properties can permit a thinner film to be used in the construction of the containment devices, particularly reversible chemical thermometers, where the profile and shape of the device is important for placement in the mouth, anus, or under the arm of persons who need their temperature taken.

Another advantage of the present invention is the ability to formulate the thermally sensitive materials under very tight tolerances because of greater assurance that the aromatic compound will remain in the cavity of the thermometer over duration of storage, including storage that could include variations in temperature during shipment and the like. The present invention would also allow repeated melting and recrystallization (“firing”) with reduced migration of chemical components outside of cavities.

Further features and advantages will be identified when reviewing embodiments of the invention in relation to the following drawings.

### BRIEF DESCRIPTION OF DRAWINGS

FIG. 1 is a plan view of a typical chemical thermometer that now includes a barrier film of the present invention, and

FIG. 2 is an enlarged, fragmented cross-section of that chemical thermometer taken along lines 2-2 in FIG. 1.

FIG. 3 is a plan view of an alternative embodiment of the chemical thermometer seen in FIG. 1.

### Embodiments of Invention

### Chemical Composition

U.S. Pat. Nos. 4,232,552; 4,299,727; 4,345,470; and 4,397,570, (all Hof et al.) and European Patent Publication 0 684 463 (A1) (Hof) and related copending, coassigned, U.S. Pat. Appln. Ser. No. 08/425,162 (Hof) now allowed, the disclosures of all of which are incorporated by reference herein in their entirety, identify many possible chemical compositions for use in single use, clinical thermometers. Each of these compositions are theoretically reversible with differing degrees of effort and energy required.

Many of these chemical compositions include halogenated aromatic compounds, such as ortho-chloronitrobenzene and ortho-bromonitrobenzene which are presently preferred as the principal ingredients of the thermally sensitive materials.

References in the Hof et al. patents and the Hof USA application and Hof EPO publication to polyisobutylene should be deemed to include any composition of polybutene that has at least a minor component of polyisobutylene. The ability of commercial manufacturers to provide pure polyisobutylene is not yet proven. Those skilled in the art will recognize that, often as a shorthand expression, references to a polymer composition allows for the possibility of other isomers of a desired polymeric composition to also be present.

Of all of these compositions, the reversible chemical compositions identified in European Patent Publication 0 684 463 (A1) (Hof) and related copending, coassigned, U.S. Pat. Appln. Ser. No. 08/425,162 (Hof) now allowed are particularly preferred. For emphasis, these compositions are an emulsion of a thermally sensitive material, means for observing a change in state of the material such as a dye, and a matrix forming material in which the thermally sensitive material is dispersed, where the composition of the thermally sensitive material is altered with calibration to a predetermined temperature. The typical formulation identified in the table of components and quantities in the Examples section of the Hof USA application and EPO publication are more particularly preferred.

Therefore, a reversible chemical composition comprises a thermally sensitive material dispersed in a matrix forming material wherein the matrix forming material is insoluble in and inert to the thermally sensitive material. But the thermally sensitive material is a mixture of valuable halogenated aromatic compounds that are susceptible to migration and must be contained in all phases.

The composition is preferably reversibly responsive to changes in temperature at a predetermined temperature. The composition can further comprise a means for visually observing a change in state of the thermally sensitive material from a solid to a liquid substantially at a predetermined temperature. The composition more particularly includes a dye or an organic moiety as the means for visual observing.

The matrix forming material can be selected from polybutylene isomers, low density polyethylene, amorphous polypropylene, and mixtures thereof, and preferably comprising at least a minor component of polyisobutylene.

More particularly, the thermally sensitive material comprises a solid solution of o-chloronitrobenzene and o-bromonitrobenzene, preferably with a minor amount of a nucleating agent. The nucleating agent can comprise an anthraquinone. Most preferably, the means for visually observing is an organic moiety selected from the group consisting of pinacyanol iodide, a mixture of ethyl red and bromophenol red, a mixture of ethyl red and bromocresolpurple, and a mixture of ethyl red and bromophenol blue.

Optionally the thermally sensitive material includes a compatible thickener. “Compatible Thickener” refers to the compatibility of the thickener with the remainder of the chemical composition of the present invention. Different color change dyes can have different compatibilities with different thickeners.

For example, it has been observed with the Hof et al. European Publication compositions that relative incompatibility is found in the change of the color of the solid temperature indicating compositions of matter with the dye as having a color change in the solid from a rose/tan solid to a bluish solid. This change in color can mask the actual change from rose/tan solid to a blue liquid, the actual solid/liquid color contrast that is important in observing the result of a temperature measurement. Therefore, compatibility of a thickener within a given chemical composition is needed to minimize color changes without phase change, in order to maximize the appearance of changes in solid-to-liquid upon which the chemical thermometry is founded.

Moreover, compatibility is concentration dependent. Higher concentrations of a thickener will produce more of a color aberration than lower concentrations. Very high concentrations of thickener do begin to discolor the chemical composition.

An effective amount of thickener is determined by the degree of thermal abuse to which the thermometers are expected to be exposed. The determination of an effective amount for the preferred thickeners of the present invention occurred by challenge testing to the likely commercial conditions of use. More particularly, the challenge testing comprised about 5-7 cycles of slow heating and cooling (the most deleterious of possible usage conditions) from room temperature to about 50° C. and about 4-5 weeks of continuous storage at that elevated temperature.

It should be understood by those skilled in the art that less aggressive challenge testing for less dramatic possible usage conditions could provide a more expanded list of compatible thickeners.

Optionally, the choice of thickeners includes fumed silicas which have been reacted with reagents to modify their surfaces with attached moieties. Most of the reagents are silicone derivatives, preferably also containing a basic nitrogen group. The nitrogen group (or other basic group) contributes to compatibility with the preferred organic moieties as the means for visually observing a change in state of the thermally sensitive material because the organic moiety has basic characteristics also, i.e., pinacyanol iodide is a basic dye.

While a surface modified silica having basic characteristics is presently preferred, the invention is not so limited. If one skilled in the art desired to employ a different means for observing that contained more acidic characteristics, then the preferred surface modified silica could be selected from those thickeners that have acidic characteristics.

Based on this selection criteria for a compatible thickener, a presently preferred compatible thickener is a silane-surface-modified fumed silica thickener sold by Degussa Corporation of Ridgefield Park, N.J., USA under the trade designation of “Aerosil™ R-504 Fumed Silica”. In this instance, the surface modification is provided by triethoxy-propyl-amino silanes and hexamethyl-di-silazanes.

The amount of compatible thickener can range from about 0.14 to about 1.5 weight percent of the total chemical composition, and preferably from about 0.70 to about 1.41 weight percent.

Also optionally, one can include a contrast enhancing agent in the matrix forming material. Contrast enhancing agents can be dye-based inks or toners depending the extent of miscibility of the solvent and the extent of inertness of the dye(s) with the Reversible Chemical Composition identified above. Nonlimiting examples of contrast enhancing agents include Oil Red O and Oil Red EGN dyes from commercial laboratory supply companies such as Aldrich Chemical Co. and ACROS Chemical Company (formerly Kodak Chemical). Particularly preferred as a contrast enhancing agent is the Oil Red EGN dye.

The amount of contrast enhancing agent can range from about 0.003 to about 0.01 weight percent of the total chemical composition, and preferably from about 0.006 to about 0.009 weight percent.

### Barrier Film

Having addressed the types of chemical compositions possible for containment within a device such as cavities of a chemical thermometer, a discussion of the importance of the barrier film and its embodiments is now possible. Because of the precision needed for the chemicals to melt at exactly the correct temperatures, it is imperative that an exact containment system be developed. This must perform at elevated temperatures (i.e. temperatures which will induce a phase change in the chemical compositions), as well as sustain longer thermal abuse cycles.

Nonlimiting examples of barrier films useful in devices of the present invention are fluorinated hydrocarbon compositions such as FEP (fluorinated ethylene-propylene), PFA (perfluoroalkyl-tetrafluoroethylene), PVDF (polyvinylidene fluoride), ECTFE (ethylene-chlorotrifluoroethylene), PCTFE (polychlorotrifluoroethylene), ETFE (ethylene-tetrafluoroethylene) and the like. FEP, and PFA are commercially available from DuPont of Wilmington, Del., USA; PVDF and ECTFE are commercially available from Westlake Plastics, of Lenni, Pa., USA. ETFE is commercially available from Allied Signal of Chicago, Ill., USA. The thickness of the barrier films of the present invention can range from about 0.012 mm to about 0.072 mm and preferably from about 0.024 mm to about 0.048 mm.

The barrier film can be adhered to the other components of a device such as a chemical thermometer, by using adhesives such as those pressure sensitive adhesives disclosed in Satas, Ed., Handbook of Pressure Sensitive Adhesives, Second Edition, (Van Nostrand, N.Y., 1989), the disclosure of which is incorporated by reference herein. If there is a concern about compatibility of the adhesive with the thermally sensitive material, one can use polyisobutylene as the adhesive according to the disclosure of U.S. Pat. No. 4,397,570 (Hof et al.). Preferably, the barrier film is treated with a surface treatment such as corona treatments known to those skilled in the art. More preferably, the commercial sources provide such surface treated films to avoid the necessity of treating such films separately at greater expense. The treatment improves the adhesion of the barrier film (one or both major surfaces) to the remainder of the chemical thermometer.

### Chemical Thermometer

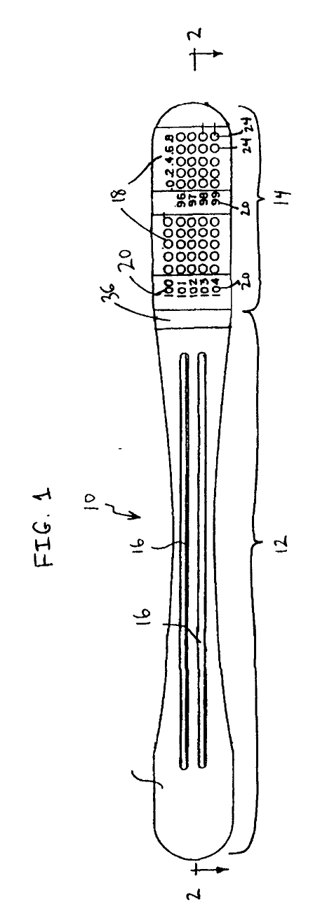

FIG. 1 shows a plan view of a substantially flat thermometer for measuring temperatures at 0.36° C. (0.2° F.) increments from 35.3° C to 40.4° C. (96.0° F. to 104.8° F.), using the compositions disclosed in copending, coassigned, U.S. patent applicatio Ser. No. 08/425,162 (Hof) now allowed. FIG. 1 shows an “hourglass” shaped thermometer 10 having two different portions at opposing ends: a handling portion 12 and a sensing portion 14. On the handling portion 12, there are plurality of reinforcing ribs 16 in the narrowest region of the hourglass shape. On the sensing portion 14 is an array 18 of cavities, each of which contains a different chemical composition in the increments described above. If the thermometer is intended to measure temperature in ° C., then typically 50 cavities are used. Otherwise the cavities for ° F. thermometers have 45 cavities. If some temperature region other than the human body is to be measured, the number of cavities can be adjusted according to the needs of those skilled in the art. Indicia 20 marks the range of temperatures to be recorded in the various cavities in the array 18.

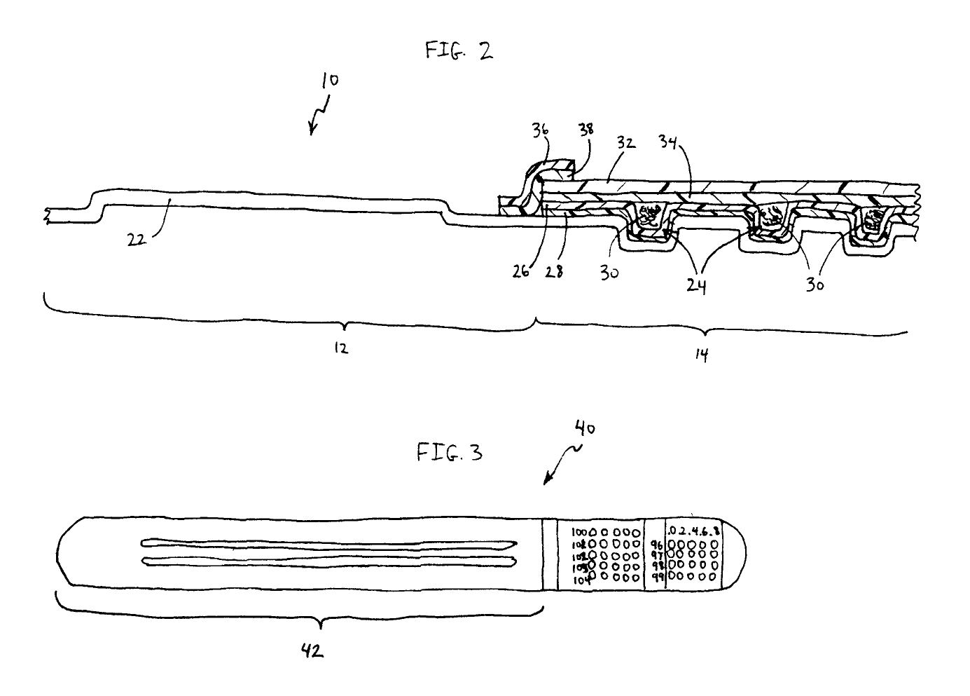

FIG. 2 is a cross-section of thermometer 10 taken along lines 2--2 in FIG. 1 to show the various layers of thermometer 10. A polymeric base layer 22 extends across both handling portion 12 and sensing portion 14. Each cavity 24 is created in layer 22. Lining at least the sensing portion 14 of layer 22, especially into and adjacent to each cavity 24, is a barrier film layer 26 adhered to such layer 22 by an adhesive layer 28. Into each cavity 24 is placed a quantity 30 of reversible thermometer composition, each cavity containing a different composition. Over the sensing portion 14 and especially the cavities 24 containing compositions 26, a transparent cover sheet 32 is adhered using an adhesive layer 34. To assure that layers 22, 26, 28, 32, and 34 do not delaminate during storage or use, optionally, a tape comprising a backing 36 and an adhesive layer 38 is applied across the width of the intersection between the handle portion 12 and the sensing portion 14 where layers 26, 28, 32, and 34 terminate.

FIG. 3 is an alternative embodiment of a chemical thermometer which has a different shape from that conventional shape as seen in FIG. 1. Thermometer 40 has a handling portion 42 and a sensing portion that are indistinct in variation in width. Whereas FIG. 1 shows a thermometer that has an “hourglass” shape between the area of the cavities and the opposite end of the thermometer, FIG. 3 shows a thermometer in a shape similar to a tongue depressor, a common shape known to patients visiting doctors' offices. This shape can be described as having substantially parallel side borders along the entire length of the thermometer, in contrast to the “hourglass” shape of the thermometer seen in FIG. 1 and the Hof patents identified above. Not only is this “substantially parallel side” shape convenient and recognizable to users of the chemical thermometer, but also this shape has performance, manufacturing and economic advantages. Specifically, the substantially parallel side embodiment provides a stiffer, yet flexible implement that can enter the mouth, axilla, or anus without discomfort. Specifically, the substantially parallel side embodiment minimizes waste of materials during manufacturing because adjoining thermometers during manufacturing need not have any material separating them before separation into individual thermometers if such thermometers were made using die cut processes known to those skilled in the art. Nonlimiting examples of such processes include using punch press die cutting equipment with intermittent action or rotary processing equipment. These processes are generally disclosed in U.S. Pat. No. 1,400,002 (Roger); U.S. Pat. No. 4,795,516 (Strand); U.S. Pat. No. 4,817,261 (Sessions et al.); U.S. Pat. No. 4,909,885 (Swenson); and U.S. Pat. No. 5,531,855 (Heinecke et al.); and PCT Patent Publication WO 98/02089 (Loutis et al.) with a die configured to cut in the shape of the “substantially parallel side” embodiment of the chemical thermometer.

Nonlimiting examples of polymeric films useful for layer 22 include polyolefins, polyesters, polyvinyl chlorides and like, with polyethylene terephthalate being preferred because of its ability to be formed with ribs 16 and an array 18 of cavities and because of its low cost. Layer 22 can have a thickness ranging from about 0.1 mm to about 0.2 mm and preferably about 0.15 mm.

Barrier film layer 26 can use any of the barrier films identified above and can have a thickness in this embodiment ranging from about 0.01 mm to about 0.04 mm and preferably about 0.02 mm.

Adhesive layer 28 can be any adhesive that is capable of reliably adhering barrier film layer 26 to polymeric layer 22 and especially in cavities 24 where compositions 30 reside. Nonlimiting examples of such adhesives include acrylate pressure sensitive adhesive commercially available from Minnesota Mining and Manufacturing Company of St. Paul, Minn., USA as transfer adhesives, hot melt adhesives commercially available from H.B. Fuller Company of St. Paul, Minn., USA, and urethane containing adhesives commercially available from Morton Thiokol of Chicago, Ill., USA suitable for adhesion to transparent packaging films, such as SN-393C adhesive. The acceptable and preferred thicknesses of such adhesive layer 28 is the same as for the barrier film layer 26 to minimize the profile of the laminate, especially in the cavities 24.

Compositions 30 in thermometer 10 can be any of the compositions for reversible temperature sensing described above. These compositions 30 can be placed in cavities 24 using the methods described in copending, coassigned, U.S. patent application Ser. No. 08/845,671 (Focarino), the disclosure of which is incorporated by reference herein. Briefly, the apparatus for metering and delivering a precise quantity of an emulsion composition to a surface, comprising: (a) at least one cartridge having a volume containing the emulsion composition; (b) at least one piston associated with each cartridge for collapsing the volume under a controlled pressure; (c) at least one tubular needle for receiving a flow of emulsion composition from each cartridge; (d) a fluid connection between each tubular needle and each cartridge for delivering the emulsion composition from each cartridge to each needle; and (e) a valve for each fluid connection.

The cover sheet 32 can be any polymeric material that is reasonably transparent for viewing of the array 18 of cavities 24 and provides a mechanical and chemical barrier for the migration or escape of compositions 30 from cavities 24. Nonlimiting examples of polymeric materials include any of the barrier films described above, polyolefins, polyesters, or polyvinyl chlorides. Polyethylene terephthalate is presently preferred. The acceptable and preferred thicknesses of sheet 32 match those of the barrier film layer 26.

Adhesive layer 34 can be any of the adhesives disclosed in U.S. Pat. No. 4,397,570 (Hof et al.), the disclosure of which is incorporated by reference herein. The acceptable and preferable thicknesses of adhesive layer 34 match those of the barrier film layer 26.

Optional tape backing 36 can be any of the polymers useful for base layer 22 and can be a polypropylene. Again to minimize profile of tape at the termini of layers 26, 28, 32, and 34, the acceptable and preferred thicknesses of backing match those of barrier film layer 26.

Optional tape backing adhesive layer 38 can be any pressure sensitive adhesive disclosed in those pressure sensitive adhesives disclosed in Satas, Ed., Handbook of Pressure Sensitive Adhesives Second Edition, (Van Nostrand, N.Y., 1989), the disclosure of which is incorporated by reference herein. Again to minimize profile of tape at the termini of layers 26, 28, 32, and 34, the acceptable and preferred thicknesses of backing match those of barrier film layer 26. Preferably, the adhesive is an acrylate-based pressure sensitive adhesive.

### Usefulness of the Invention

The barrier film of the present invention assists in the storage of the superior reversible chemical thermometers disclosed in copending, coassigned, U.S. Patent application Ser. No. 08/425,162 (Hof). The shapes of the chemical thermometer can range from the conventional shape seen in FIG. 1 and the new shape seen in FIG. 3.

Examples provide further explanation of the embodiments of this invention.

### EXAMPLES

Samples of various fluorinated compounds were used as potential barrier films. The barrier films tried were FEP (fluorinated ethylene-propylene), PFA (perfluoroalkoxy-tetrafluoroethylene), PCTFE (polychlorotrifluoroethylene), ECTFE (ethylenechlorotrifluoroethylene), PVDF (polyvinylidene fluoride), PCTFE (chlorotrifluoroethylene), and ETFE (ethylenetetrafluoroethylene). All of the films except PVDF provided superior barrier properties. PVDF provided comparable barrier properties to prior materials such as nylon but with a thinner cross-section. These films were cut, allowed to dry overnight in oven at 90° C., and weighed. A mixture (o-bromonitrobenzene, o-chloronitrobenzene, polyisobutylene/polyisobutene, red EGN dye, pinacyanol chloride, anthraquinone and a fumed silica as disclosed above) was then placed over a measured area of the polymer samples. These constructions were covered and heated at 90° C. for one week. At this point, they were uncovered, delicately and consistently dried off, and weighed again. A visual observation was also made of the face-down portion of film to see if anything had permeated clear through. The weight gain could then be attributed to the absorption of chemical into the film.

It was discovered that FEP and PFA provided the best, consistent results for lack of absorption over several replicates of polymer type and manufacturer. The average weight change of PFA and FEP was noticeably less (about 50%) than other fluorinated liners, as shown in the following data. By comparison control films of nylon, Surlyn brand ionomer, and PE/nylon were also tested and had approximately 10 times greater weight gain than any of the fluorinated films of the present invention. Table 1 shows the results.

TABLE 1

| | | Original | Final | | |
|---|---|---|---|---|---|
| | | Weight | Weight | Weight | Avg. |
| Example | Film | (g) | (g) | Change | Change |
| 1 | FEP (0.025 mm) | 0.3012 | 0.3000 | −0.0012 | 5 × 10⁻⁵ |
| 2 | FEP (0.025 mm) | 0.2992 | 0.3005 | 0.0013 | |
| 3 | ECTFE (0.025 mm) | 0.2066 | 0.2073 | 0.0007 | 0.00075 |
| 4 | ECTFE (0.025 mm) | 0.2686 | 0.2694 | 0.0008 | |
| 5 | PFA (0.025 mm) | 0.2948 | 0.2948 | 0 | 0.0003 |
| 6 | PFA (0.025 mm) | 0.3078 | 0.3084 | 0.0006 | |
| A | Surlyn ™ | 1.7139 | 1.7170 | 0.0031 | 0.0028 |
| | Ionomer/PET | | | | |
| | Composite | | | | |
| | (0.05/0.15 mm) | | | | |
| B | Surlyn ™ | 1.7134 | 1.7159 | 0.0025 | |
| | Ionomer/PET | | | | |
| | Composite Brand | | | | |
| | Ionomer | | | | |
| | (0.05/0.15 mm) | | | | |
| C | Nylon/PET | 1.5727 | 1.5753 | 0.0026 | 0.0024 |
| | (0.05/0.15 mm) | | | | |
| D | Nylon/PET | 1.5647 | 1.5669 | 0.0022 | |
| | (0.05/0.15 mm) | | | | |
| E | Polyethylene/Nylon/ | 1.6486 | 1.6500 | 0.0014 | 0.0014 |
| | PET | | | | |
| | (0.0125/0.05/ | | | | |
| | 0.15 mm) | | | | |
| F | Polyethylene/Nylon/ | | | | |
| | PET | | | | |
| | (0.0125/0.05/ | | | | |
| | 0.15 mm) | | | | |

REMEASURED AFTER ABOUT 3 HOURS

| | | | | | |
|---|---|---|---|---|---|
| 1′ | FEP (0.025mm) | 0.3012 | 0.3000 | −0.0012 | −0.0002 |
| 2′ | FEP (0.025 mm) | 0.2992 | 0.3000 | 0.0008 | |
| 3′ | ECTFE (0.025 mm) | 0.2066 | 0.2074 | 0.0008 | 0.00085 |
| 4′ | ECTFE (0.025 mm) | 0.2686 | 0.2695 | 0.0009 | |
| 5′ | PFA (0.025 mm) | 0.2948 | 0.2947 | −1E−04 | 0.0003 |
| 6′ | PFA (0.025 mm) | 0.3078 | 0.3085 | 0.0007 | |
| A′ | Surlyn ™ Ionomer/ | 1.7139 | 1.7171 | 0.0032 | 0.0028 |
| | PET (0.05/0.15 mm) | | | | |
| B′ | Surlyn ™ Ionomer/ | 1.7134 | 1.7158 | 0.0024 | |
| | PET | | | | |
| | (0.05/0.15 mm) | | | | |
| C′ | Nylon/PET | 1.5727 | 1.5788 | 0.0061 | 0.00605 |
| | (0.05/0.15 mm) | | | | |
| D′ | Nylon/PET | 1.5647 | 1.5707 | 0.006 | |
| | (0.05/0.15 mm) | | | | |
| E′ | Polyethylene/Nylon/ | 1.6486 | 1.6506 | 0.002 | 0.0022 |
| | PET | | | | |
| | (0.0125/0.05/ | | | | |
| | 0.15 mm) | | | | |
| F′ | Polyethylene/Nylon/ | 1.6375 | 1.6399 | 0.0024 | |
| | PET | | | | |
| | (0.0125/0.05/ | | | | |
| | 0.15 mm) | | | | |

As noted in the test results above (confirmed by later replicates), the films containing fluorinated hydrocarbons, specifically FEP and PFA, were not as likely to absorb elements of the chemical composition as were the previously used barrier materials.

An additional point of note is that over a matter of a few hours, the nylon samples gained weight without exposure to the chemical compositions. As is familiar to those experienced in the art, polyamides such as nylon readily absorb a fraction of moisture, even in ambient conditions. Although nylon is commonly seen as a good barrier, perhaps this moisture absorption provides enough of pathway through the nylon for the chemical composition—an effect to which the fluorinated hydrocarbons are not susceptible.

FEP, PFA, PE/nylon, nylon and Surlyn films were then placed in a 2×3 central composite factorial experiment in complete construction thermometers. All of these constructions were then subjected to accelerated aging (5 weeks at 90° C.) and shipping abuse testing (one cycle being a 4 hour ramp from ambient to 120° C., soak for 4 hours at 120° C., and 4 hour ramp to ambient). Testing in water baths were then taken of samples of each construction. Results indicated that the fluorinated liners were able to maintain accuracy for 3 weeks of accelerated aging and 4 shipping abuse cycles. Nylon was able to survive 2-3 weeks and 2-3 cycles, and the other films did not survive 2 weeks or 2 cycles. This confirmed the conclusions based on data seen in Table 1 and shows that the active ingredients (ortho-chloronitrobenzene and ortho-bromonitrobenzene) are being better contained in the fluorinated liners. This test was repeated for reproducibility with the same results.

Due to the poor performance of the PE/nylon construction relative to the nylon-only construction, it can additionally be proposed that the PE layer is providing an alternative pathway for the movement of the chemical composition. This is confirmed in examination of the thermometers, which show discolorations around the dots of these thermometers. Those skilled in the art will know that polyolefinic layers such as PE are readily susceptible to movement of aromatic compounds and that the above explanation correlates to that understanding.

The invention is not limited to these embodiments. The claims follow.

What is claimed is:

1. A barrier film for halogenated aromatic compounds, comprising:
a fluorinated hydrocarbon composition selected from the group consisting of fluorinated ethylene-propylene, perfluoroalkyl-tetrafluoroethylene, polyvinylidene fluoride, ethylene-chlorotrifluoroethylene, polychlorotrifluoroethylene, ethylene-tetrafluoroethylene, and combinations thereof.

2. The barrier film of claim 1, wherein the film has a thickness ranging from 0.012 mm to about 0.072 mm.

3. The barrier film of claim 1, wherein the film has a thickness ranging from 0.024 mm to about 0.048 mm.

4. A chemical thermometer, comprising:
(a) a base layer having a multiplicity of cavities defined therein to determine a like number of predetermined temperatures in a predetermined temperature range, the cavities containing a like number of different compositions of matter therein,
(b) a transparent cover sheet in sealing engagement therewith,
(c) a different thermometer composition in each cavity, and
(d) a barrier film of a fluorinated hydrocarbon composition residing between the base layer and the thermometer composition in each cavity.

5. The thermometer of claim 4, wherein the barrier film has a thickness ranging from 0.01 mm to about 0.04 mm.

6. The thermometer of claim 4, wherein the barrier film has a thickness of about 0.02 mm.

7. The thermometer of claim 4, wherein the barrier film is surface treated.

8. The thermometer of claim 4, wherein the barrier film is adhered to the base layer with an adhesive selected from the group consisting of acrylate pressure sensitive adhesives, hot melt adhesives, and urethane containing adhesives.

9. The thermometer of claim 4, wherein the thermometer has a handling portion and a sensing portion, and wherein the handling portion has substantially parallel sides.

10. The thermometer of claim 4, wherein the cavities range from about 45 to about 50.

11. A chemical thermometer, comprising:
(a) a base layer having a multiplicity of cavities defined therein to determine a like number of predetermined temperatures in a predetermined temperature range, the cavities containing a like number of different compositions of matter therein,
(b) a transparent cover sheet in sealing engagement therewith, and
(c) a different thermometer composition in each cavity, wherein the thermometer has a handling portion and a sensing portion, and wherein the handling portion has substantially parallel sides.
